(19) United States
(12) Patent Application Publication
(10) Pub. No.: US 2026/0144972 A1
(43) Pub. Date: May 28, 2026

(54) MICRONEEDLE ARRAY APPLICATOR AND SYSTEM

(71) Applicant: Kindeva Drug Delivery L.P., Woodbury, MN (US)

(72) Inventor: Richard Brewer, Loughborough (GB)

(21) Appl. No.: 19/126,987

(22) PCT Filed: Nov. 3, 2023
(86) PCT No.: PCT/US2023/036744

§ 371 Date: May 2, 2025

Related U.S. Application Data
(60) Provisional application No. 63/422,965, filed on Nov. 5, 2022.

(51) Int. Cl. A61M 37/00

(52) U.S. Cl. CPC A61M 37/0015; A61M 2037/0023; A61M 2037/0061; A61M 2205/106

(57) ABSTRACT

Various embodiments of a microneedle array applicator (12) are disclosed. The applicator includes an applicator body (16) and an opening (22) extending through the body, an actuator (26) configured to slidably engage with the applicator body between an unprimed configuration and an actuated configuration of the applicator, and a plunger (32) disposed at least partially within the opening of the applicator body. The plunger includes a latch (36) configured to engage a slot (40) disposed in an upper portion (18) of the applicator body to retain the plunger at least partially within the opening of the applicator body when the applicator is in the unprimed configuration. The applicator further includes a stored energy device (42). Axially compressing the actuator and the applicator body manipulates the applicator from the unprimed configuration to the actuated configuration, causing the actuator to inwardly deflect the latch and disengage the latch from the slot of the applicator body to release the plunger.

This application claims the benefit of U.S. Provisional Application No. 63/422,965, filed Nov. 5, 2022, the disclosure of which is incorporated by reference herein in its entirety.

### BACKGROUND

Transdermal and topical drug delivery can be used for therapeutic treatment, but the number of molecules that can be effectively delivered using these routes can be limited by the barrier properties of skin. The main barrier to transport of molecules through the skin is the stratum corneum (the outermost layer of the skin).

Different skin treatment techniques have been proposed to increase the permeability or porosity of the outermost skin layers, such as the stratum corneum, thus enhancing drug delivery through or into those layers. The stratum corneum is a complex structure of compact keratinized cell remnants separated by lipid domains. The stratum corneum is formed of keratinocytes, which includes the majority of epidermal cells, that lose their nuclei and become corneocytes. These dead cells make up the stratum corneum, which has a thickness of only about 10-30 microns and protects the body from invasion by exogenous substances and the outward migration of endogenous fluids and dissolved molecules. Various skin treatment methods include the use of microneedles, laser ablation, RF ablation, heat ablation, sonophoresis, iontophoresis, or combinations of these treatment methods.

Devices including arrays of relatively small structures, sometimes referred to as microneedles or micro-pins, have been described for delivering therapeutic agents and other substances through the skin and other surfaces. These devices can be pressed against the skin to pierce the stratum corneum such that the therapeutic agents and other substances can sequentially or simultaneously pass through that layer and into the tissues below. Microneedles of these devices pierce the stratum corneum upon contact, making a plurality of microscopic slits that serve as passageways through which molecules of active components can be delivered into the body. In delivering an active component, the microneedle device can be provided with a reservoir for temporarily retaining an active component in liquid form prior to delivering the active component through the stratum corneum. In some constructions, the microneedles can be hollow to provide a liquid flow path directly from the reservoir and through the microneedles to deliver the therapeutic substance through the skin. In alternate constructions, active component(s) may be coated on the microneedle array and delivered directly through the skin after the stratum corneum has been punctured.

Microneedle arrays and patches can be deployed with an applicator. While the microneedle arrays and patches are generally used once and then discarded, applicators can be either single-use or reusable.

### SUMMARY

In general, the present disclosure provides various embodiments of a system that includes a microneedle array and a microneedle array applicator that can be utilized to apply the array to skin of a user. The applicator can include an applicator body and an actuator that is configured to be slidably engaged with the applicator body. A plunger can be disposed at least partially within an opening disposed through the applicator body. The applicator can be configured to be manipulated from an unprimed configuration to an actuated configuration by axially compressing the actuator and the applicator body along an axis. Such compression causes the plunger to become disengaged from the applicator body. When disengaged from the applicator body, the plunger directs the microneedle array through a lower surface of the applicator body and onto the skin of the user. In one or more embodiments, the user can manipulate the applicator from the unprimed configuration to the actuated configuration in a continuous motion without the applicator pausing or being retained in a primed configuration prior to actuation.

In one aspect, the present disclosure provides a microneedle array applicator. The applicator includes an applicator body having an upper portion, a lower portion, and an opening extending through the upper portion and the lower portion along an axis, where the lower portion is configured to be disposed proximate skin of a user, an actuator configured to slidably engage with the applicator body between an unprimed configuration and an actuated configuration of the applicator; a plunger disposed at least partially within the opening of the applicator body. The plunger includes a base and a latch that extends from an inner surface of the base. The latch is configured to engage a slot disposed in the upper portion of the applicator body to retain the plunger at least partially within the opening of the applicator body when the applicator is in the unprimed configuration. The applicator further includes a stored energy device disposed between the base of the plunger and an inner surface of the actuator. The stored energy device is configured to be compressed and energized as the applicator is manipulated from the unprimed configuration to the actuated configuration. The applicator is configured such that axially compressing the actuator and the applicator body manipulates the applicator from the unprimed configuration to the actuated configuration, where manipulating the applicator from the unprimed configuration to the actuated configuration causes the actuator to inwardly deflect the latch and disengage the latch from the slot of the applicator body to release the plunger.

In another aspect, the present disclosure provides a system that includes a microneedle array and a microneedle array applicator. The applicator includes an applicator body having an upper portion, a lower portion, and an opening extending through the upper portion and the lower portion along an axis, where the lower portion is configured to be disposed proximate skin of a user; an actuator configured to slidably engage with the applicator body between an unprimed configuration and an actuated configuration of the applicator; a plunger disposed at least partially within the opening of the applicator body. The plunger includes a base and a latch that extends from an inner surface of the base. The latch is configured to engage a slot disposed in the upper portion of the applicator body to retain the plunger at least partially within the opening of the applicator body when the applicator is in the unprimed configuration. The applicator further includes a stored energy device disposed between the base of the plunger and an inner surface of the actuator. The stored energy device is configured to be compressed and energized as the applicator is manipulated from the unprimed configuration to the actuated configuration. The applicator is configured such that axially compressing the actuator and the applicator body manipulates the applicator from the unprimed configuration to the actuated configuration, where manipulating the applicator from the unprimed configuration to the actuated configuration causes the actuator to inwardly deflect the latch and disengage the latch from the slot of the applicator body to release the plunger.

In another aspect, the present disclosure provides a method that includes disposing a microneedle array within an opening in an applicator body of an applicator, where the opening extends through an upper portion and a lower portion of the applicator body along an axis, and where the applicator further includes an actuator slidably engaged with the applicator body and a plunger disposed at least partially within the opening of the applicator body. The method further includes disposing the applicator proximate skin of a user, and manipulating the applicator from an unprimed configuration to an actuated configuration. Manipulating the applicator from the unprimed configuration to the actuated configuration includes axially compressing the actuator of the applicator and the applicator body along the axis. A stored energy device disposed between a base of the plunger and an inner surface of the actuator is compressed and energized when the applicator is manipulated from the unprimed configuration to the actuated configuration. Further, the actuator inwardly defects a latch that extends from an inner surface of a base of the plunger and disengages the latch from a slot disposed in the upper portion of the applicator body to release the plunger.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

Throughout the specification, reference is made to the appended drawings, where like reference numerals designate like elements, and wherein:

FIG. 1 is a schematic perspective view of one embodiment of a system that includes a microneedle array and a microneedle array applicator.

FIG. 2 is a schematic exploded view of the system of FIG. 1.

FIG. 3 is a schematic cross-section view of the system of FIG. 1 with the applicator of the system in an unprimed configuration.

FIG. 4 is a schematic cross-section view of the system of FIG. 1 with the applicator being actuated from the unprimed configuration to an actuated configuration.

FIG. 5 is a schematic cross-section view of the system of FIG. 1 with the applicator in the actuated configuration.

FIG. 6 is a schematic top plan view of an applicator body of the applicator of FIG. 1.

FIG. 7 is a schematic bottom plan view of the applicator body of the applicator of FIG. 1.

FIG. 8 is a schematic bottom plan view of an actuator of the applicator of FIG. 1.

FIG. 9 is a perspective view of the microneedle array of the system of FIG. 1.

FIG. 10 is a schematic cross-section view of a portion of the microneedle array of FIG. 9.

FIG. 11 is a schematic perspective view of a stored energy device of the applicator of FIG. 1.

FIG. 12 is a flowchart of one technique of utilizing the system of FIG. 1.

### DETAILED DESCRIPTION

In general, the present disclosure provides various embodiments of a system that includes a microneedle array and a microneedle array applicator that can be utilized to apply the array to skin of a user. The applicator can include an applicator body and an actuator that is configured to be slidably engaged with the applicator body. A plunger can be disposed at least partially within an opening disposed through the applicator body. The applicator can be configured to be manipulated from an unprimed configuration to an actuated configuration by axially compressing the actuator and the applicator body along an axis. Such compression causes the plunger to become disengaged from the applicator body When disengaged from the applicator body, the plunger directs the microneedle array through the lower surface of the applicator body and onto the skin of the user. In one or more embodiments, the user can manipulate the applicator from the unprimed configuration to the actuated configuration in a continuous motion without the applicator pausing or being retained in a primed configuration prior to actuation.

Before any embodiments of the present disclosure are explained in detail, it is to be understood that these embodiments are not limited in application to the details of construction and the arrangement of components set forth in the description or illustrated in the drawings. The embodiments are capable of being practiced or of being carried out in various ways. Also, it is to be understood that the phraseology and terminology used herein is for the purpose of description and should not be regarded as limiting. Further, terms such as “front,” “rear,” “top,” “bottom,” “upward,” “downward,” “under,” and the like are only used to describe elements as they relate to one another, but are in no way meant to recite specific orientations of these elements, to indicate or imply necessary or required orientations of the such elements, or to specify how these elements that are described herein will be used, mounted, displayed, or positioned in use.

In discussing the microneedle array applicators of the present disclosure, the term “downward,” and variations thereof, is sometimes used to describe the direction in which microneedles are pressed into skin, and “upward” to describe the opposite direction. Those of skill in the art, however, will understand that the applicators can be used where the microneedles are pressed into skin at an angle to the direction of the earth's gravity, or even in a direction contrary to that of the earth's gravity, and that these terms are only used for simplicity and clarity to describe relative directions.

The term “transdermally,” and variations thereof, is generally used to refer to any type of delivery of an active ingredient that crosses any portion of skin. That is, transdermally can generally include systemic delivery (i.e., where the active ingredient is transported across, or substantially through, the dermis such that the active ingredient is delivered into the bloodstream), as well as intradermal delivery (i.e., where the active ingredient is transported partially through the dermis, e.g., across the outer layer (stratum corneum) of the skin, where the active ingredient is delivered into the skin. That is, transdermal delivery as used herein includes delivery of an active ingredient that is transported across at least a portion of skin (but not necessarily all of the layers of skin), rather than merely being topically applied to an outer layer of the skin.

The present disclosure generally relates to an applicator and technique or method for applying a microneedle device, including an array of microneedles, to skin (or a biological membrane) to treat the skin (i.e., create small holes or perforations or micropores in the skin) and/or to deliver an active agent to the skin.

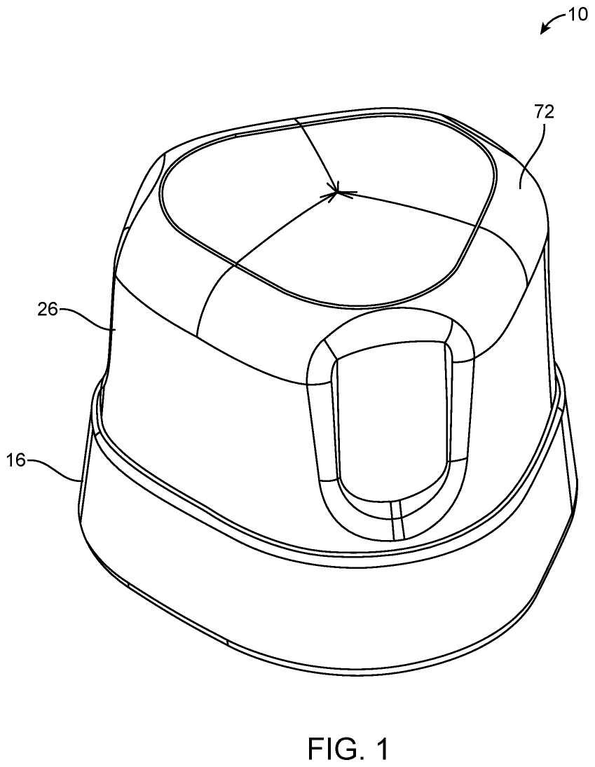

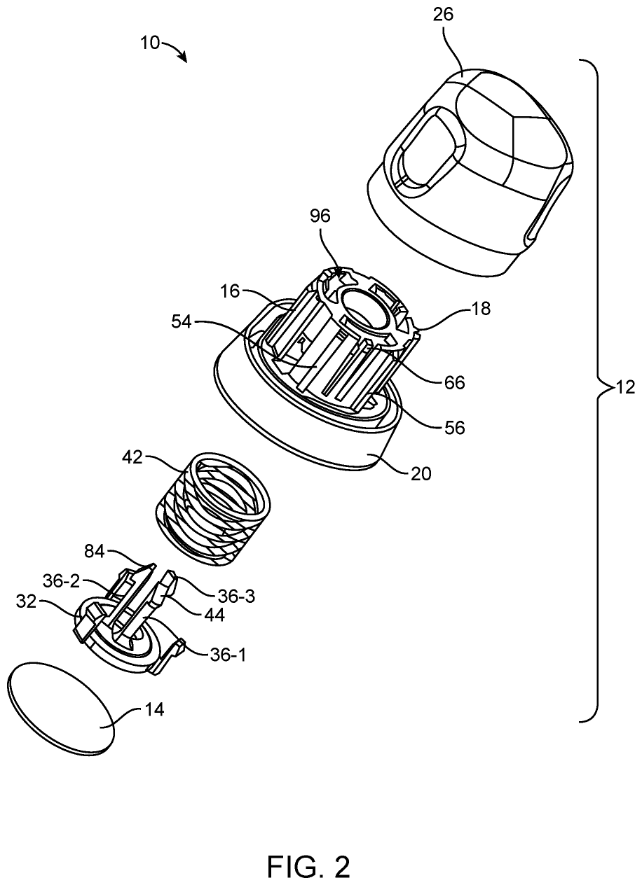

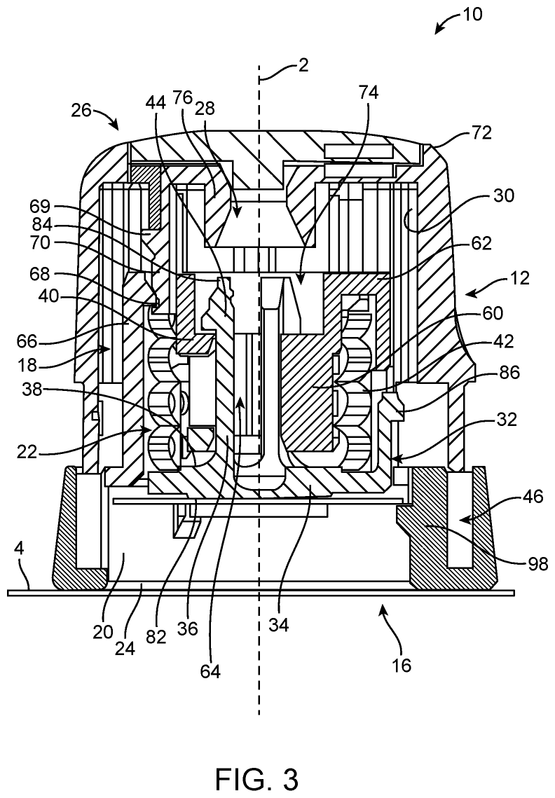

FIGS. 1-11 are various views of one embodiment of a microneedle array applicator system 10 and elements or components of such system. The system 10 includes a microneedle array applicator 12 and a microneedle array 14 (FIG. 2). As can be seen in FIG. 3, the applicator 12 includes an applicator body 16 having an upper portion 18, a lower portion 20, and an opening 22 extending through the upper portion and the lower portion along an axis 2. The lower portion 20 is configured to be disposed proximate skin 4 of a user. The applicator 12 further includes an actuator 26 configured to slidably engage with the applicator body 16 between an unprimed configuration (FIG. 3) and an actuated configuration (FIG. 5) of the applicator. The applicator 12 further includes a plunger 32 disposed at least partially within the opening 22 of the applicator body 16. The plunger 32 includes a base 34 and a latch 36 that extends from an inner surface 38 of the base. The latch 36 is configured to engage a slot 40 disposed in the upper portion 18 of the applicator body 16 to retain the plunger at least partially within the opening 22 of the applicator body when the applicator 12 is in the unprimed configuration. The applicator 12 further includes a stored energy device 42 disposed between the base 34 of the plunger 32 and an inner surface 30 of the actuator 26. The stored energy device 42 is configured to be compressed and energized as the applicator 12 is manipulated from the unprimed configuration to the actuated configuration.

The applicator 12 is configured such that axially compressing the actuator 26 and the applicator body 16 manipulates the applicator from the unprimed configuration to the actuated configuration. Further, manipulating the applicator 12 from the unprimed configuration to the actuated configuration causes the actuator to inwardly deflect the latch 36 and disengage the latch from the slot 40 of the applicator body 16 to release the plunger 32.

The applicator body 16 of the applicator 12 can include any suitable material, e.g., at least one inorganic (e.g., metallic) or organic (e.g., polymeric) material. Further, the applicator body 16 can take any suitable shape and have any suitable dimensions. For example, the applicator body 16 can have a uniform cross-sectional shape in a plane orthogonal to the axis 2. In one or more embodiments, the applicator body 16 can have a cross-sectional shape that varies along the axis 2. Further, the upper portion 18 of the applicator body 16 can have a cross-sectional shape that is different from a cross-sectional shape of the lower portion 20.

The upper portion 18 can have an elliptical cross-sectional shape in the plane orthogonal to the axis 2. As shown in FIG. 2, the upper portion 18 includes a cylindrical shape that extends from the lower portion 20 of the applicator body 16. Further, the lower portion 20 can have an elliptical cross-sectional shape in the plane orthogonal to the axis 2. In one or more embodiments, the lower portion 20 of the applicator body 16 includes a rounded triangular shape (FIG. 7) in the plane orthogonal to the axis 2.

The applicator body 16 can include a recess 46 (FIG. 5) disposed in the lower portion 20 of the applicator body that is configured to receive a lower portion 48 of the actuator 26. Further, a ridge or rib 52 can be disposed on the actuator 26 that is configured to engage the applicator body 16 when the applicator 12 is in the actuated configuration (FIG. 5), where the rib acts as a stop to motion of the actuator when the actuator and applicator body are compressed.

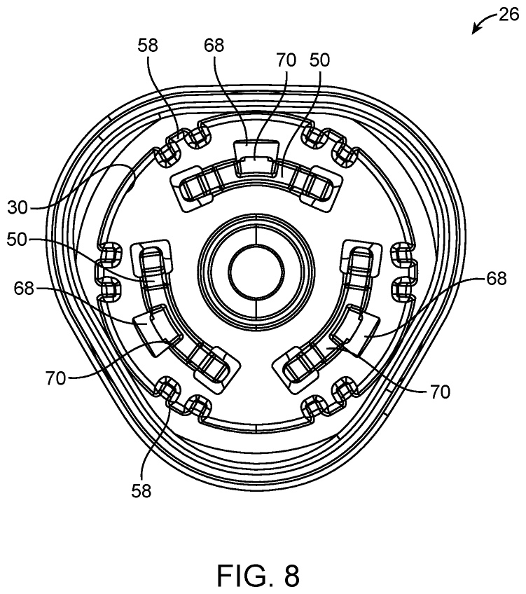

The applicator body 16 can further include one or more ribs 54 (FIGS. 2 and 7) disposed on an outer surface 56 of the upper portion 18 of the applicator body that extend in a direction substantially parallel to the axis 2. Each rib 54 is configured to slidably engage a slot 58 (FIG. 8) disposed in the inner surface 30 of the actuator 26 to prevent the actuator from rotating about the axis 2 when the actuator is manipulated along the axis relative to the applicator body 16. In one or more embodiments, one or more ribs 54 can be disposed on the inner surface 30 of the actuator 26, and one or more slots 58 can be disposed in the outer surface 56 of the upper portion 18 of the applicator body 16 that slidably engage each rib.

The applicator body 16 can further include one or more upper tabs 66 (FIG. 3) that can take any suitable shape and be disposed in any suitable portion or portions of the upper portion 18 of the applicator body. As shown in FIG. 3, the upper tab 66 can be disposed in the upper portion 18 of the applicator body 16 and extend in a direction substantially parallel to the axis 2. The applicator body 16 further include one or more apertures 96 disposed in the upper portion 18 proximate the upper tab 66 that is adapted to receive an actuator arm 70 that extends from the inner surface 30 of the actuator 26. The actuator arm 70, which can define a portion of the inner surface 30 of the actuator, is configured to engage the upper tab 66 of the applicator body 16 to retain the actuator 26 to the applicator body such that the applicator body and actuator remain connected after assembly. During such assembly, the applicator body 16 can be slid into the actuator 26 such that actuator arm 70 is inserted into the aperture 96 and engages the upper tab 66. In one or more embodiments, the actuator arm 70 include a notch 68 (FIG. 3) that engages the upper tab 66 of the applicator body 16 and retains the actuator 26 to the applicator body when the applicator is in the unprimed configuration. The actuator arm 70 can further include a ramped portion 69 that is configured to engage the upper tab 66 of the applicator body 16 and retain the applicator in the actuated configuration (FIG. 5) such that the user cannot reset the applicator 12 in the unprimed configuration.

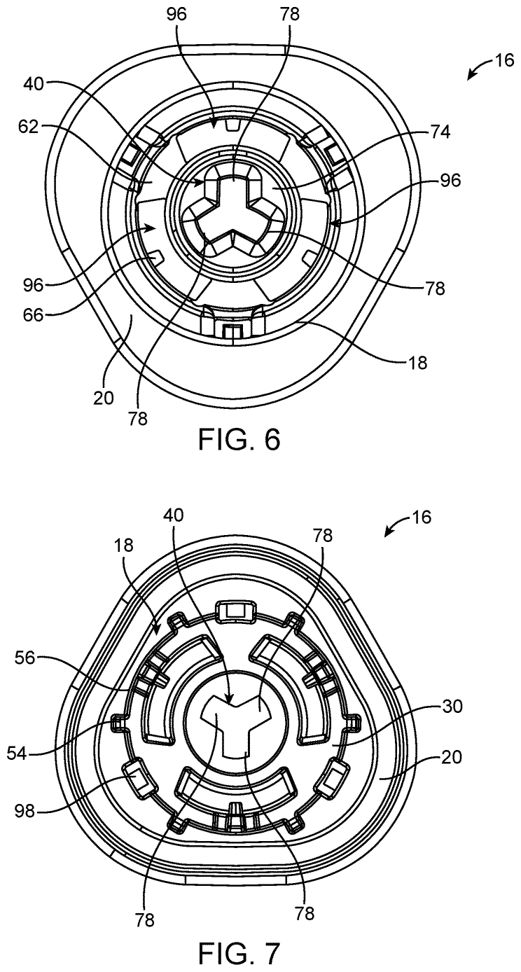

The slot 40 of the applicator body 16 can be disposed in the upper portion 18 of the applicator body. In one or more embodiments, the slot 40 can be disposed in an upper surface 62 of the upper portion 18 of the applicator body 16. The slot 40 can take any suitable shape and have any suitable dimensions. As shown in FIG. 7, the slot 40 includes branches 78 each configured to receive a latch 36 of the plunger 32 and retain the plunger 32 at least partially within the opening 22 of the applicator body 16 when the applicator is in the unprimed configuration (FIG. 3). The applicator body 16 can include any suitable number of slots 40. The slot 40 can be disposed in any suitable portion or portions of the upper surface 62 of the upper portion 18 of the applicator body 16. In one or more embodiments, the upper surface 62 of the upper portion 18 can include a recessed portion 74 (FIG. 6) in which the slot 40 can be disposed. The recessed portion 74 can take any suitable shape and have any suitable dimensions. In one or more embodiments, the recessed portion 74 can have a selected depth such that the latch 36 of the plunger 32 does not extend beyond the upper surface 62 of the applicator body 16 when the latch is disposed through the slot 40. Further, the recessed portion 74 can take a shape that is configured to receive an extension 76 that extends from the inner surface 30 of the actuator 26 when the applicator 12 is in the actuated configuration as shown in FIG. 5.

The opening 22 of the applicator body 16 extends through the upper portion 18 and the lower portion 20 along the axis 2. Such opening 22 can take any suitable cross-sectional shape in the plane orthogonal to the axis 2. In one or more embodiments, a plunger guide 60 can extend into the opening 22 from the upper surface 62 of the applicator body 16. An aperture 64 can be disposed in the plunger guide 60 along the axis. Such aperture 64 can take any suitable shape. The aperture 64 terminates at the upper surface 62 of the applicator body 16 and is connected to the slot 40. The aperture 64 is configured to receive one or more latches 36 of the plunger 32. The plunger guide 60 and aperture 64 can be configured to guide the latches 36 of the plunger 32 through the slot 40 when the applicator 12 is assembled and downward toward a lower surface 24 of the lower portion 20 of the applicator body 16 when the applicator 12 is manipulated from the unprimed configuration to the actuated configuration.

The applicator body 16 further includes the lower surface 24 that is configured to be disposed proximate the skin 4 of the user. The lower surface 24 can take any suitable shape. Further, the lower surface 24 can include a textured surface or one or more protuberances that are configured to prevent the applicator 12 from sliding on the skin 4 while the applicator is being manipulated from the unprimed position to the actuated position as is further described herein.

Slidably engaged with the applicator body 16 is the actuator 26. The actuator 26 can include any suitable material, e.g., the same materials described herein regarding the applicator body 16. In one or more embodiments, the actuator 26 can be manufactured using the same material as the material of the applicator body 16. In one or more embodiments, the actuator 26 can include a material that is different from the material of the applicator body 16.

The actuator 26 can take any suitable shape and have any suitable dimensions. In one or more embodiments, an outer surface 72 of the actuator can include an ergonomic shape that is configured to be gripped by a hand of the user. As shown in FIGS. 3-5, the actuator 26 is configured to slidably engage the applicator body 16 by sliding over the applicator body such that one or more portions of the inner surface 30 of the actuator are in contact with one or more portions of the outer surface 56 of the applicator body. In one or more embodiments, the actuator 26 is configured to slide within the applicator body 16 such that one or more portions of the outer surface 72 of the actuator are in contact with one or more portions of the applicator body. In other words, the actuator 26 can be configured to slide within the applicator body 16.

In one or more embodiments, the actuator 26 is configured to slidably engage with at least the upper portion 18 of the applicator body along the axis 2. In one or more embodiments, the actuator 26 can be configured to slidably engage with the upper portion 18 and the lower portion 20 of the applicator body 16. The actuator 26 can slidably engage the applicator body 16 along the axis 2 between the unprimed configuration (FIG. 3) and the actuated configuration (FIG. 5) of the applicator 12 as is further described herein.

The actuator 26 can be configured to inwardly deflect the latch 36 and disengage the latch from the slot 40 of the applicator body 16 to release the plunger 32. The inner surface 30 of the actuator 26 can include any suitable elements or components that can be configured to deflect the latch 36. For example, the inner surface 30 can include a cavity 28 that is disposed in the inner surface along the axis 2. The cavity 28 of the actuator 26 can be disposed in an extension 76 that defines a portion of the inner surface 30 of the actuator 26. The cavity 28 can take any suitable shape and have any suitable dimensions. In one or more embodiments, the cavity 28 can take a frustoconical shape. In one or more embodiments, the cavity 28 is configured to inwardly deflect the latch 36 of the plunger 32 and disengage the latch from the slot 40 of the applicator body 16 to release the plunger. In one or more embodiments, the stored energy device 42 drives the plunger 32 towards the lower surface 24 of the lower portion 20 of the applicator body 16.

The inner surface 30 of the actuator 26 can further include the actuator arm 70. The actuator arm 70 can take any suitable shape and have any suitable dimensions. The actuator 26 can include any suitable number of arms 70. As shown in FIG. 8, the actuator 26 includes three actuator arms 70 that extend from the inner surface 30 of the actuator and help to define the inner surface. At least one actuator arm 70 includes the notch 68 that is configured to engage the upper tab 66 of the applicator body 16 and retain the actuator to the actuator body when the applicator 12 is in the unprimed configuration. At least one actuator arm 70 further includes the ramped portion 69 that is configured to engage the upper tab 66 of the applicator body 16 and retain the applicator 12 in the actuated configuration such that the user cannot further manipulate the applicator to the unprimed configuration. The actuator arms 70 are also configured to engage with the stored energy device 42 and compress the stored energy device between the actuator 26 and the plunger 32 as is further described herein. In one or more embodiments, each actuator arm 70 can include a contact surface 50 that is configured to contact the stored energy device 42.

Disposed at least partially within the opening 22 of the applicator body 16 is the plunger 32. The plunger 32 includes the base 34 and one or more latches 36 that extend from the inner surface 38 of the base. The latch 36 is configured to engage the slot 40 disposed in the upper portion of the applicator body 16 to retain the plunger 32 at least partially within the opening 22 of the applicator body when the applicator 12 is in the unprimed configuration.

The plunger 32 can include any suitable material, e.g., the same material described herein regarding the applicator body 16. In one or more embodiments, the plunger 32 can include a material that is different from the material of at least one of the applicator body 16 or the actuator 26. The plunger 32 can also take any suitable shape and have any suitable dimensions. Further, the base 34 can take any suitable shape and have any suitable dimensions. The base 34 includes the inner surface 38 and an outer surface 82. The outer surface 82 can be configured to contact the microneedle array 14 and drive the array onto the skin 4 when the actuator 26 is manipulated from the unprimed configuration to the actuated configuration. The outer surface 82 can be disposed in any suitable relationship relative to the microneedle array 14. In one or more embodiments, the outer surface 82 can be disposed a selected distance from the microneedle array 14 when the actuator is in the unprimed configuration. In one or more embodiments, the outer surface 82 can be in contact with the array 14 when the actuator is in the unprimed configuration

In general, the plunger 32 is disposed at least partially within the opening 22 of the applicator body 16. In one or more embodiments, the plunger 32 is disposed completely or entirely within the opening 22 of the applicator body 16 when the applicator is in the unprimed configuration. In one or more embodiments, the plunger 32 can be completely disposed within the opening 22 of the applicator body 16 when the applicator is in the actuated configuration. In one or more embodiments, at least a portion of the plunger 32 can be disposed outside of the opening 22 of the applicator body 16 when the applicator is in the actuated configuration. As shown in FIG. 5, at least a portion of the base 34 of the plunger 32 (e.g., the outer surface 82) can extend through the lower surface 24 of the lower portion 20 of the applicator body 16 when the applicator 12 is in the actuated configuration.

Extending from the inner surface 38 of the plunger 32 are the one or more latches 36. The plunger 32 can include any suitable number of latches 36. As shown in FIG. 2, the plunger 32 includes a first latch 36-1, a second latch 36-2, and third latch 36-3 (collectively latches 36) that extend from the inner surface 38 of the base 34. Each latch 36 can take any suitable shape and have any suitable dimensions. Further each latch 36 can include a ramped portion 44 disposed proximate a distal end 84 of the latch 36. The ramped portion 44 can also take any suitable shape and have any suitable dimensions. In one or more embodiments, the latches 36 can be disposed on the applicator body 16, and the slot 40 can be disposed in the plunger 32.

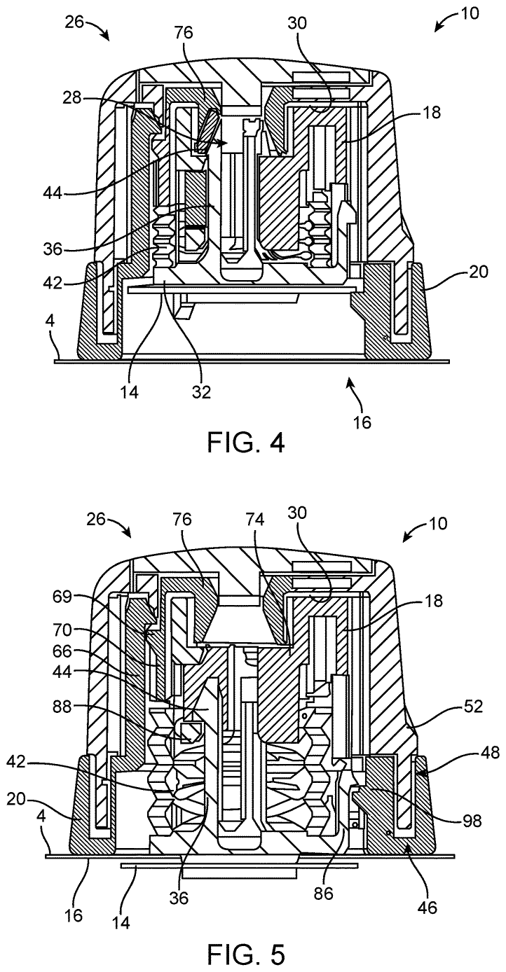

As mentioned herein, the latch 36 is configured to engage the slot 40 disposed in the upper surface 62 of the applicator body 16. The latch 36 is further configured to retain the plunger 32 at least partially within the opening of the applicator body 16 when the applicator 12 is in the unprimed configuration. Each of the first, second, and third latches 36 is configured to engage the slot 40 disposed in the upper surface 62 of the upper portion 18 of the applicator body 16 to retain the plunger at least partially within the opening 22 of the applicator body when the applicator 12 is in the unprimed configuration. Further, manipulating the actuator 26 from the primed configuration to the actuated configuration causes the actuator to inwardly deflect each of the first, second, and third latches 36 and disengage such latches from the slot 40 to release the plunger 32. FIG. 4 is a schematic cross-section view of the applicator 12 while it is being manipulated from the unprimed configuration to the actuated configuration. As can be seen in FIG. 4, the cavity 28 engages the latches 36 as the actuator 26 is compressed with the applicator body 16. As the actuator 26 continues to be compressed, the cavity 28 inwardly deflects the ramped portion 44 of each of the first, second, and third latches 36 such that they disengage from the slot and travel through the aperture 64 of the plunger guide 60.

In one or more embodiments, the plunger 32 can also include an arm 86 (FIG. 3) that extends from the base 34. The arm 86 can be configured to engage one or more projections 98 (FIG. 5) disposed in the applicator body 16 and retain the plunger to the applicator body when the applicator 12 is in the actuated configuration as shown in FIG. 5. In other words, the arm 86 retains the plunger 32 at least partially within the opening 22 of the applicator body 16 after the latch 36 has been released from engagement with the slot 40 of the upper portion 18 of the applicator body. The latches 36 of the plunger 32 can also be configured to engage a surface 88 (FIG. 5) disposed within the plunger guide aperture 64 and maintain connection of the plunger with the applicator body 16 after the applicator has been manipulated to the actuated configuration.

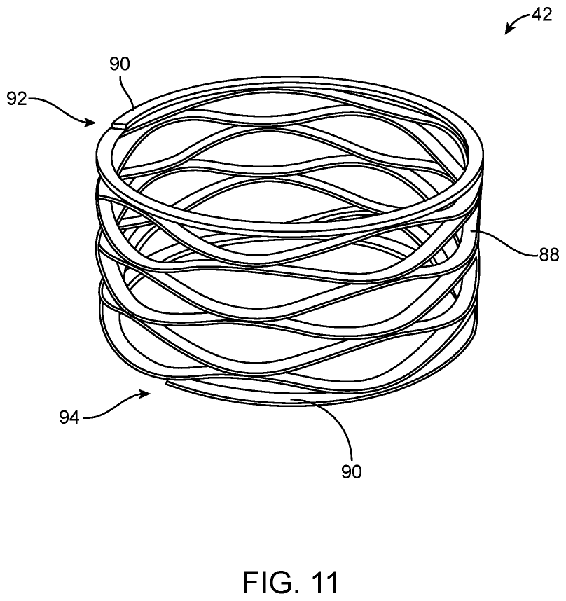

The stored energy device 42 of the applicator 12 is disposed between the base 34 of the plunger 32 and the inner surface 30 of the actuator 26 as shown in FIG. 3. In one or more embodiments, the stored energy device 42 can be disposed over the plunger guide 60. The stored energy device 42 is configured to be compressed and energized as the applicator 12 is manipulated from the unprimed configuration (FIG. 3) to the actuated configuration (FIG. 5). In one or more embodiments, the stored energy device 42 can also be configured to be slightly compressed when the applicator is in the unprimed configuration (FIG. 3) such that the device and the plunger 32 remain stable in the unprimed configuration. The stored energy device 42 can include any suitable device that is configured to store energy until such time as the applicator 12 is manipulated to the actuated configuration. When the applicator 12 is in the actuated configuration, the stored energy device 42 is configured to transfer stored energy to the plunger 32 by driving the plunger towards the lower surface 24 of the lower portion 20 of the applicator body 16. The stored energy device 42 can include one or more discrete springs that are configured in either series or parallel configurations. In one or more embodiments, the stored energy device 42 includes at least one of a conical coil spring, a coil spring, or a tension spring. In one or more embodiments, the stored energy device 42 includes a wave spring as shown in FIG. 11. In general, a wave spring is a compression spring made of flat wire and that has multiple “waves” 88 of spring per turn. In one or more embodiments, one or more shims 90 can be connected to at least a top end 92 or a bottom end 94 of the stored energy device 42 as shown in FIG. 11. Such shims 90 can provide flat surfaces at each end 92, 94 of the device 42 to interact with the inner surface 30 of the actuator 26 and the inner surface 38 of the plunger 32. In one or more embodiments, wave springs can provide an advantage over conventional coil springs as they have a lower profile when fully compressed, meaning the overall device can be smaller in height. A further advantage of a wave spring over conventional springs is that it can provide a more evenly distributed force around its diameter as there are effectively multiple springs in parallel.

The stored energy device 42 can be compressed and energized using any suitable technique. In one or more embodiments, the one or more actuator arms 70 that extend from the inner surface 30 of the actuator 26 can each be disposed within an aperture 96 (FIG. 6) disposed in the upper surface 62 of the upper portion 18 of the applicator body 16. As the actuator 26 is manipulated from the unprimed configuration to the actuated configuration, each actuator arm 70 presses against the stored energy device 42 and compresses it against the inner surface 38 of the base 34 of the plunger 32 (e.g., against the inner surface 38 of the base). Because the plunger 32 is retained in position by the latch 36, the stored energy device 42 continues to be compressed and energized until the latch is released from the slot 40 of the applicator body 16 by the actuator 26 and the plunger is driven by the stored energy device toward the lower surface 24 of the lower portion 18 of the applicator body 16.

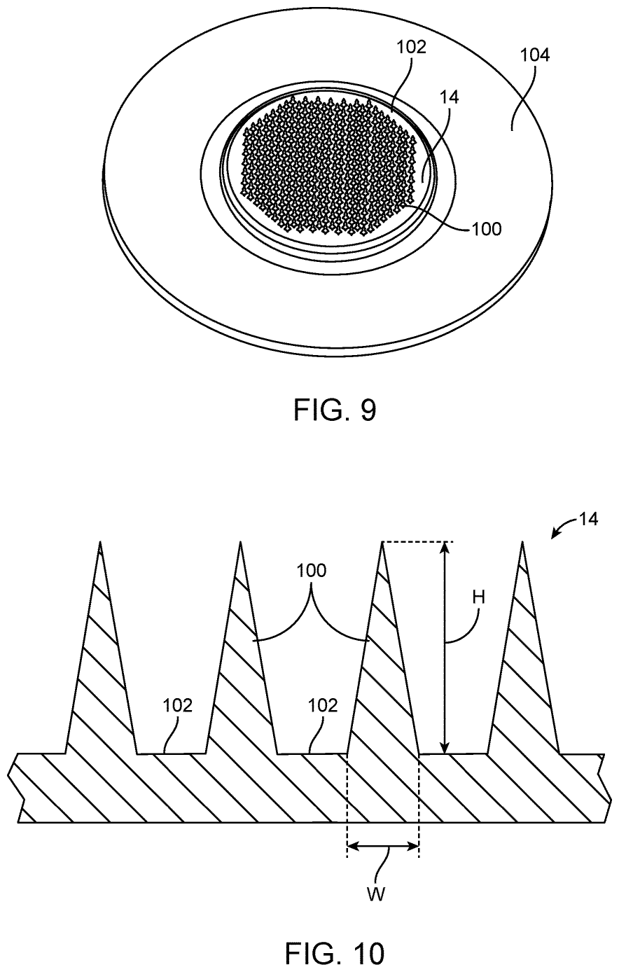

Disposed within the opening 22 of the lower portion 20 of the applicator body 16 is the microneedle array 14, which can include any suitable microneedle device or patch. For example, FIG. 9 is a schematic perspective view of one embodiment of the microneedle array 14. The microneedle array 14, which can also be referred to herein as a “microneedle device” or “patch,” can include microneedles 100 and any supporting structure or substrate 102 used to support the microneedles or couple the microneedles to other structures or components. The microneedle array 14 can be retained within the opening 22 of the applicator body 16 using any suitable technique. In one or more embodiments, the microneedle array 14 can be retained within the opening by the one or more projections 98 (FIG. 3) that extend from the lower portion 20 of the actuator body 16 into the opening 22 until the plunger 32 directs the array through the lower surface 24 of the lower portion 20 of the applicator body when the applicator is manipulated to the actuated configuration.

As mentioned herein, in one or more embodiments, active ingredients or agents (e.g., drugs) can be delivered via the microneedles 100 (e.g., via solid, dissolvable, or hollow microneedles, as described below). Examples of pharmaceutically active agents (also referred to as “drugs”) that can be incorporated into the applicators of the present disclosure are those capable of local or systemic effect when administered to the skin. Some examples include buprenorphine, clonidine, diclofenac, estradiol, granisetron, isosorbide dinitrate, levonorgestrel, lidocaine, methylphenidate, nicotine, nitroglycerine, oxybutynin, rivastigmine, rotigotine, scopolamine, selegiline, testosterone, tulobuterol, and fentanyl, which are commercially available in the form of transdermal devices. Other examples include antiinflammatory drugs, both steroidal (e.g., hydrocortisone, prednisolone, triamcinolone) and nonsteroidal (e.g., naproxen, piroxicam); bacteriostatic agents (e.g., chlorhexidine, hexylresorcinol); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotazoals (e.g., metronidazole); antifungals (e.g., nystatin), coronary vasodilators; calcium channel blockers (e.g., nifedipine, diltiazem); bronchodilators (e.g., theophylline, pirbuterol, salmeterol, isoproterenol), enzyme inhibitors such as collagenase inhibitors, protease inhibitors, acetylcholinesterase inhibitors (e.g., donepezil), elastase inhibitors, lipoxygenase inhibitors (e.g., A64077), and angiotensin converting enzyme inhibitors (e.g., captopril, lisinopril); other antihypertensives (e.g., propranolol); leukotriene antagonists (e.g., ICI204,219); anti-ulceratives such as H2 antagonists; steroidal hormones (e.g., progesterone); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinolin-4-amine, N-[4-(4-amino-2 -ethyl-1H-imidazo[4,5-c]quinolin-1-yl)butyl]methanesulfonamide, and acyclovir); local anesthetics (e.g., benzocaine, propofol, tetracaine, prilocaine); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl citrate, sufentanil, hydromorphone hydrochloride); peptide hormones (e.g., human or animal growth hormones, LHRH, parathyroid hormones); cardioactive products such as atriopeptides; antidiabetic agents (e.g., insulin, exanatide); enzymes (e.g., anti-plaque enzymes, lysozyme, dextranase); antinauseants, anticonvulsants (e.g., carbamazine); immunosuppressives (e.g., cyclosporine); psychotherapeutics (e.g., diazepam); sedatives (e.g., phenobarbital); anticoagulants (e.g., heparin, enoxaparina sodium); analgesics (e.g., acetaminophen); antimigraine agents (e.g., ergotamine, melatonin, sumatriptan, zolmitriptan); antiarrhythmic agents (e.g., flecainide); antiemetics (e.g., metaclopromide, ondansetron, granisetron hydrochloride); anticancer agents (e.g., methotrexate); neurologic agents such as anxiolytic drugs; hemostatics; anti-obesity agents; dopamine agonists (e.g., apomorphine); GnRH agonists (e.g., leuprolide, goserelin, nafarelin); fertility hormones (e.g., hCG, hMG, urofollitropin); interferons (e.g., interferon-alpha, interferon-beta, interferon-gamma, pegylated interferon-alpha), and the like, as well as pharmaceutically acceptable salts and esters thereof. The amount of drug that constitutes a therapeutically effective amount can be readily determined by those skilled in the art with due consideration of the particular drug, the particular carrier, and the desired therapeutic effect.

In one or more embodiments, peptide therapeutic agents (natural, synthetic, or recombinant) can be delivered via the microneedles 100 (e.g., via solid, dissolvable, or hollow microneedles, as described below). Examples of peptide therapeutic agents that can be incorporated into the applicators of the present disclosure include parathyroid hormone (PTH), parathyroid hormone related protein (PTHrP), calcitonin, lysozyme, insulin, insulinotropic analogs, glatiramer acetate, goserelin acetate, somatostatin, octreotide, leuprolide, vasopressin, desmopressin, thymosin alpha-1, atrial natriuretic peptide (ANP), endorphin, vascular endothelial growth factor (VEGF), fibroblast-growth factor (FGF), erythropoietin (EPO), bone morphogenetic proteins (BMPs), epidermal growth factor (EFG), granulocyte colony-stimulating factor (G-CSF), granulocyte macrophage colony stimulating factor (GM-CSF), insulin-like growth factor (IGF), platelet-derived growth factor (PDGF), growth hormone release hormone (GHRH), dornase alfa, tissue plasminogen activator (tPA), urokinase, ANP clearance inhibitors, lutenizing hormone releasing hormone (LHRH), melanocyte stimulating hormones (alpha & beta MSH), pituitary hormones (hGH), adrenocorticotropic hormone (ACTH), human chorionic gonadotropin (hCG), streptokinase, interleukins (e.g. IL-2, IL-4, IL-10, IL-12, IL-15, IL-18), protein C, protein S, angiotensin, angiogenin, endothelins, pentigetide, brain natriuretic peptide (BNP), neuropeptide Y, islet amyloid polypeptide (IAPP), vasoactive intestinal peptide (VIP), hirudin, glucagon, oxytocin, and derivatives of any of the foregoing peptide therapeutic agents.

In one or more embodiments, drugs that are of a large molecular weight may be delivered transdermally. Increasing molecular weight of a drug typically can cause a decrease in unassisted transdermal delivery. Examples of such large molecules include proteins, peptides, nucleotide sequences, monoclonal antibodies, vaccines, polysaccharides, such as heparin, and antibiotics, such as ceftriaxone. Examples of suitable vaccines include therapeutic cancer vaccines, anthrax vaccine, flu vaccine, Lyme disease vaccine, rabies vaccine, measles vaccine, mumps vaccine, chicken pox vaccine, small pox vaccine, hepatitis vaccine, hepatitis A vaccine, hepatitis B vaccine, hepatitis C vaccine, pertussis vaccine, rubella vaccine, diphtheria vaccine, encephalitis vaccine, Japanese encephalitis vaccine, respiratory syncytial virus vaccine, yellow fever vaccine, recombinant protein vaccine, DNA vaccines, polio vaccine, herpes vaccine, human papilloma virus vaccine, pneumococcal vaccine, meningitis vaccine, whooping cough vaccine, tetanus vaccine, typhoid fever vaccine, cholera vaccine, tuberculosis vaccine, severe acute respiratory syndrome (SARS) vaccine, HSV-1 vaccine, HSV-2 vaccine, HIV vaccine and combinations thereof. The term “vaccine” thus includes, without limitation, antigens in the forms of proteins, polysaccharides, oligosaccharides, or weakened or killed viruses. Additional examples of suitable vaccines and vaccine adjuvants are described in U.S. Patent Application Publication No. 2004/0049150 (Dalton et al.), the disclosure of which is hereby incorporated by reference in its entirety.

In another embodiment, small-molecule drugs that are otherwise difficult or impossible to deliver by passive transdermal delivery may be used. Examples of such molecules include salt forms; ionic molecules, such as bisphosphonates, including sodium alendronate or pamedronate; and molecules with physicochemical properties that are not conducive to passive transdermal delivery.

Microneedles 100 useful for practicing the present disclosure can have a variety of configurations and features, such as those described in the following patents and patent applications, the disclosures of which are incorporated herein by reference in their entirety. One embodiment for the microneedles 100 includes the structures disclosed in U.S. Patent Application Publication No. 2005/0261631 (Clarke et al.), which describes microneedles having a truncated tapered shape and a controlled aspect ratio. Another embodiment for the microneedles includes the structures disclosed in U.S. Pat. No. 6,091,975 (Daddona et al.), which describes blade-like microprotrusions for piercing the skin. Still another embodiment for the microneedles includes the structures disclosed in U.S. Pat. No. 6,312,612 (Sherman et al.), which describes tapered structures having a hollow central channel. Yet still another embodiment for the microneedles includes the structures disclosed in U.S. Pat. No. 6,379,324 (Gartstein et al.), which describes hollow microneedles having at least one longitudinal blade at the top surface of the tip of the microneedle. A further embodiment for the microneedles includes the structures disclosed in U.S. Patent Application Publication Nos. US2012/0123387(Gonzalez et al.) and US2011/0213335 (Burton et al.), which both describe hollow microneedles. A still further embodiment for the microneedles includes the structures disclosed in U.S. Pat. No. 6,558,361 (Yeshurun) and 7,648,484 (Yeshurun et al.), which both describe hollow microneedle arrays and methods of manufacturing thereof.

Various embodiments of microneedles that can be employed in the microneedle arrays of the present disclosure are described in PCT Publication No. WO2012/074576 (Duan et al.), which describes liquid crystalline polymer (LCP) microneedles; and PCT Publication No. WO2012/122162 (Zhang et al.), which describes a variety of different types and compositions of microneedles that can be employed in the microneedles of the present disclosure.

In one or more embodiments, the microneedle material can be (or include) silicon, glass, or a metal such as stainless steel, titanium, or nickel titanium alloy. In one or more embodiments, the microneedle material can be (or include) a polymeric material, preferably a medical grade polymeric material. Exemplary types of medical grade polymeric materials include polycarbonate, liquid crystalline polymer (LCP), polyether ether ketone (PEEK), cyclic olefin copolymer (COC), polybutylene terephthalate (PBT). Preferred types of medical grade polymeric materials include polycarbonate and LCP.

In one or more embodiments, the microneedle material can be (or include) a biodegradable polymeric material, preferably a medical grade biodegradable polymeric material. Exemplary types of medical grade biodegradable materials include polylactic acid (PLA), polyglycolic acid (PGA), PGA and PLA copolymer, polyester-amide polymer (PEA).

In one or more embodiments, the microneedles can be a prepared from a dissolvable, degradable, or disintegradable material referred to herein as “dissolvable microneedles”. A dissolvable, degradable, or disintegradable material is any solid material that dissolves, degrades, or disintegrates during use. In particular, a “dissolvable microneedle” dissolves, degrades, or disintegrates sufficiently in the tissue underlying the stratum corneum to allow a therapeutic agent to be released into the tissue. The therapeutic agent may be coated on or incorporated into a dissolvable microneedle. In one or more embodiments, the dissolvable material is selected from a carbohydrate or a sugar. In one or more embodiments, the dissolvable material is polyvinyl pyrrolidone (PVP). In one or more embodiments, the dissolvable material is selected from the group consisting of hyaluronic acid, carboxymethylcellulose, hydroxypropylmethylcellulose, methylcellulose, polyvinyl alcohol, sucrose, glucose, dextran, trehalose, maltodextrin, and a combination thereof.

In one or more embodiments, the microneedles can be made from (or include) a combination of two or more of any of the above-mentioned materials. For example, the tip of a microneedle may be a dissolvable material, while the remainder of the microneedle is a medical grade polymeric material.

A microneedle or the plurality of microneedles in a microneedle array useful for practicing the present disclosure can have a variety of shapes that are capable of piercing the stratum corneum. In one or more embodiments, one or more of the plurality of microneedles can have a square pyramidal shape, triangular pyramidal shape, stepped pyramidal shape, conical shape, microblade shape, or the shape of a hypodermic needle. In one or more embodiments, one or more of the plurality of microneedles can have a square pyramidal shape. In one or more embodiments, one or more of the plurality of microneedles can have a triangular pyramidal shape. In one or more embodiments, one or more of the plurality of microneedles can have a stepped pyramidal shape. In one or more embodiments, one or more of the plurality of microneedles can have a conical shape. In one or more embodiments, one or more of the plurality of microneedles can have a microblade shape. In one or more embodiments, one or more of the plurality of microneedles can have the shape of a hypodermic needle. The shape can be symmetric or asymmetric. The shape can be truncated (for example, the plurality of microneedles can have a truncated pyramid shape or truncated cone shape). In a preferred embodiment, the plurality of microneedles in a microneedle array each have a square pyramidal shape.

In one or more embodiments, the plurality of microneedles in a microneedle array are solid microneedles (that is, the microneedles are solid throughout). In one or more embodiments, the plurality of solid microneedles in a solid microneedle array can have a square pyramidal shape, triangular pyramidal shape, stepped pyramidal shape, conical shape, or microblade shape. In a preferred embodiment, the plurality of solid microneedles in a solid microneedle array each have a square pyramidal shape.

In one or more embodiments, the plurality of microneedles in a microneedle array are hollow microneedles (that is, the microneedles contain a hollow bore through the microneedle). The hollow bore can be from the applicator body of the microneedle to the tip of the microneedle or the bore can be from the applicator body of the microneedle to a position offset from the tip of the microneedle. In one or more embodiments, one or more of the plurality of hollow microneedles in a hollow microneedle array can have a conical shape, cylindrical shape, square pyramidal shape, triangular pyramidal shape, or the shape of a hypodermic needle.

In one or more embodiments, one or more of the plurality of hollow microneedles in a hollow microneedle array can have a conical shape. In one or more embodiments, one or more of the plurality of hollow microneedles in a hollow microneedle array can have a cylindrical shape. In one or more embodiments, one or more of the plurality of hollow microneedles in a hollow microneedle array can have a square pyramidal shape. In one or more embodiments, one or more of the plurality of hollow microneedles in a hollow microneedle array can have a triangular pyramidal shape. In one or more embodiments, one or more of the plurality of hollow microneedles in a hollow microneedle array can have the shape of a hypodermic needle. In a preferred embodiment, the plurality of hollow microneedles in a hollow microneedle array each have the shape of a conventional hypodermic needle.

FIG. 10 shows a portion of the microneedle array 14 that includes four microneedles 100 positioned on the substrate 102. Each microneedle 100 has a height h, which is the length from the tip of the microneedle to the microneedle body at the substrate 102. Either the height of a single microneedle 100 or the average height of all microneedles on the microneedle array 14 can be referred to as the height of the microneedle, h. In one or more embodiments, each of the plurality of microneedles 100 (or the average of all of the plurality of microneedles) has a height of about 100 to about 3000 micrometers, in one or more embodiments, about 100 to about 1500 micrometers, in one or more embodiments, about 100 to about 1200 micrometers, and, in one or more embodiments, about 100 to about 1000 micrometers. In one or more embodiments, each of the plurality of microneedles 100 (or the average of all of the plurality of microneedles) has a height of about 200 to about 1200 micrometers, about 200 to about 1000 micrometers, about 200 to about 750 micrometers, or about 200 to about 600 micrometers.

In one or more embodiments employing solid microneedles 100, each of the plurality of solid microneedles (or the average of all of the plurality of solid microneedles) has a height of about 100 to about 1500 micrometers, about 100 to about 1200 micrometers, about 200 to about 1000 micrometers, about 200 to about 750 micrometers, about 200 to about 600 micrometers, or about 500 micrometers.

In one or more embodiments employing hollow microneedles, each of the plurality of hollow microneedles (or the average of all of the plurality of hollow microneedles) has a height of about 100 to about 3000 micrometers, about 800 to about 1400 micrometers, or about 500 micrometers.

A single microneedle or the plurality of microneedles 100 in a microneedle array can also be characterized by their aspect ratio. The aspect ratio of a microneedle is the ratio of the height of the microneedle, h to the width (at the body of the microneedle), w (as shown in FIG. 10), The aspect ratio can be presented as h: w. In one or more embodiments, each of the plurality of microneedles 100 (or the average of all the plurality of microneedles 100) has (have) an aspect ratio in the range of 2:1 to 5:1. In some of these embodiments, each of the plurality of microneedles 100 (or the average of all of the plurality of microneedles 100) has (have) an aspect ratio of at least 3:1.

In one or more embodiments, the array of microneedles 100 contains about 100 to about 1500 microneedles per cm² of the array of microneedles.

In one or more embodiments, each of the plurality of microneedles (or the average of all of the plurality of microneedles) in a microneedle array can penetrate into the skin to a depth of about 100 to about 400 micrometers, or about 100 to about 300 micrometers.

For all of the above embodiments, it will be appreciated that the depth of penetration (DOP) of each of the plurality of microneedles (or the average of all of the plurality of microneedles) in a microneedle array may not be the full length of the microneedles themselves. In one or more embodiments, the microneedle array 14 according to the present disclosure can be in the form of a patch. One example of such an embodiment is shown in FIG. 9. Microneedle array 14 includes the plurality of microneedles 100 disposed on the substrate 102, a backing layer 104, and an adhesive layer (not shown) disposed on the backing layer between the backing layer and the substrate of the microneedle array. Microneedles 100 can be arranged in any desired pattern or distributed over substrate 102 randomly. As shown, microneedles 100 are arranged in uniformly spaced rows. When arranged in rows, the rows can be arranged so that microneedles 100 are aligned or offset. In one or more embodiments (not shown), microneedles 100 can be arranged in a polygonal pattern such as a triangle, square, rectangle, pentagon, hexagon, heptagon, octagon, or trapezoid. In other embodiments (not shown), microneedles 100 can be arranged in a circular or oval pattern.

In one or more embodiments, the surface area of the substrate 102 covered with microneedles 100 is about 0.1 cm² to about 20cm². In one or more embodiments, the microneedles 100 can be disposed over substantially the entire surface of the array. In other embodiments (not shown), a portion of the substrate 102 may not be provided with microneedles (that is, a portion of the substrate is non-structured). In some of these embodiments, the non-structured surface has an area of more than about 1 percent and less than about 75 percent of the total area of the device surface that faces the skin surface. In another of these embodiments, the non-structured surface has an area of more than about 0.65 cm² (0.10 square inch) to less than about 6.5 cm² (1 square inch).

For hollow microneedles, a hollow channel or bore extends through the substrate and microneedles. In one or more embodiments, the bore exits at a channel opening at or near the tip of the hollow microneedle. The channel preferably exits at an opening near the tip of the hollow microneedle. Most preferably, the channel or bore continues along a central axis of the microneedle but exits similar to a hypodermic needle on a sloping side-wall of the microneedle to help prevent blockage of the channel by tissue upon insertion. In one or more embodiments, the diameter of the channel bore is about 10 to about 200 micrometers. In one or more embodiments of hollow microneedles, the average cross-sectional area of the channel bore is about 75 to about 32,000 micrometers. In one or more embodiments of hollow microneedle arrays, the average spacing between adjacent microneedles (as measured from microneedle tip to microneedle tip) is between about 0.7 mm and about 20 mm. In one or more embodiments of hollow microneedle arrays, the average spacing between adjacent microneedles (as measured from microneedle tip to microneedle tip) is greater than about 0.7 mm. In one or more embodiments of hollow microneedle arrays, the average spacing between adjacent microneedles is less than about 20 mm.

In one or more embodiments of solid microneedle arrays, the average spacing between adjacent microneedles (as measured from microneedle tip to microneedle tip) is between about 200 micrometers and about 2000 micrometers. In one or more embodiments of solid microneedle arrays, the average spacing between adjacent microneedles (as measured from microneedle tip to microneedle tip) is greater than about 200 micrometers. In one or more embodiments of solid microneedle arrays, the average spacing between adjacent microneedles is less than about 2000 micrometers.

The microneedle arrays can be manufactured in any suitable way such as by injection molding, compression molding, metal injection molding, stamping, photolithography, or extrusion. In one embodiment, hollow microneedle arrays can be made by injection molding of a polymer such as medical grade polycarbonate or LCP, followed by laser drilling to form the channels of the microneedles.

The system 10 can be assembled using any suitable technique. In one or more embodiments, the applicator body 16, the actuator 26, and the plunger 32 of the applicator 12 can each be manufactured using any suitable technique, e.g., molding, injection molding, 3D printing, machining, casting, etc. The actuator 26 can be slid over the applicator body 16 using any suitable technique until the upper tab 66 engages the notch 68 of the actuator arm 70 such that the upper tab retains the applicator body to the actuator. Further, the stored energy device 42 can be inserted into the opening 22 of the applicator body 16 using any suitable technique such that the device is proximate to or in contact with the actuator arm 70. The plunger 32 can be inserted into the opening 22 of the applicator body 16 using any suitable technique. In one or more embodiments, the latch 36 of the plunger 32 can be inserted through the aperture 64 of the plunger guide 60 until the ramped portion 44 of the latch is inserted through the slot 40 and retained by the slot. The microneedle array 14 can be disposed within the applicator body 16 using any suitable technique. In one or more embodiments, the microneedle array 14 can be disposed such that it rests on the one or more projections 98 that are disposed within the lower portion 20 of the applicator body 16 as shown in FIG. 3. The projections 98 can retain the microneedle array 14 within the applicator body 16 until the applicator 12 is manipulated to the actuated configuration, which causes the plunger 32 to be released and disengage the microneedle array from the projections and drive the array through the lower surface 24 of the applicator body.

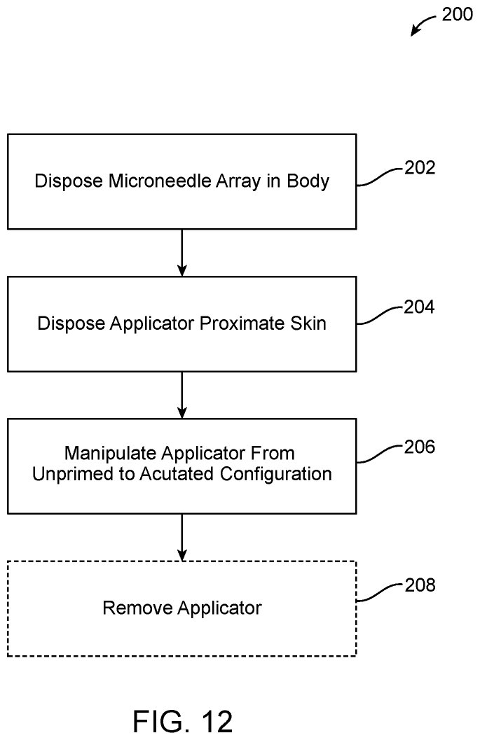

Any suitable technique can be utilized with the system 10 to deliver the microneedle array 14 to the skin 4 of the user. For example, FIG. 12 is a flowchart of one embodiment of a technique or method 200 of utilizing the system 10. Although described regarding the system 10 of FIGS. 1-11, the technique 200 can be utilized with any suitable microneedle array system. At 202, the microneedle array 14 can be disposed within the applicator body 16 of the assembled applicator 12 using any suitable technique. In one or more embodiments, the microneedle array 14 is disposed within the applicator body 16 during manufacture of the system. In one or more embodiments, the array 14 can be disposed within the applicator body 16 by the user. At 204, the lower surface 24 of the lower portion 20 of the applicator body 16 can be disposed proximate the skin 4 of the user or another surface such as a sterile or sanitary surface. In one or more embodiments, the applicator 12 can be disposed on the skin 4 of the user. At 206, the applicator 12 and be manipulated from the unprimed configuration to the actuated configuration at 106 using any suitable technique. In one or more embodiments, the applicator 12 can be manipulated by axially compressing the actuator 26 of the applicator and the applicator body 16 along the axis 2. As the actuator 26 and the applicator body 16 are compressed, the stored energy device 42 is compressed and energized by the actuator. Further, during actuation, the actuator 26 inwardly defects the one or more latches 36 and disengages the latches from the slot 40 disposed in the upper portion 18 of the applicator body 16 to release the plunger 32. In one or more embodiments, the stored energy device 42 drives the plunger 32 towards the lower surface 24 of the lower portion 20 of the applicator body 16. Manipulating the applicator 12 from the unprimed configuration to the actuated configuration causes the plunger 32 to drive the microneedle array 14 from the projections 98 of the inner surface of the lower portion 20 of the applicator body 16 and toward the skin 4 of the user such that the microneedle array is delivered to the skin. The applicator 12 is configured to apply the microneedle array 14 to the skin 4 with the intended velocity to achieve a desired array needle depth of penetration for efficient drug delivery. In one or more embodiments, the user can manipulate the applicator 12 from the unprimed configuration to the actuated configuration in one continuous motion without the applicator pausing or being retained in a primed configuration. At 208, the applicator 12 can optionally be removed from the skin 4 after the microneedle array 14 has been delivered to the skin 4.

In one or more embodiments, the user may release the actuator 26 prior to actuation of the applicator 12. In such embodiments, the stored energy device 42 will return the applicator to the unprimed configuration as shown in FIG. 3.

The invention is defined in the claims. However, below there is provided a non-exhaustive listing of non-limiting examples. Any one or more of the features of these examples may be combined with any one or more features of another example, embodiment, or aspect described herein

Example Ex1. A microneedle array applicator. The applicator includes an applicator body having an upper portion, a lower portion, and an opening extending through the upper portion and the lower portion along an axis, where the lower portion is configured to be disposed proximate skin of a user; an actuator configured to slidably engage with the applicator body between an unprimed configuration and an actuated configuration of the applicator; a plunger disposed at least partially within the opening of the applicator body. The plunger includes a base and a latch that extends from an inner surface of the base. The latch is configured to engage a slot disposed in the upper portion of the applicator body to retain the plunger at least partially within the opening of the applicator body when the applicator is in the unprimed configuration. The applicator further includes a stored energy device disposed between the base of the plunger and an inner surface of the actuator. The stored energy device is configured to be compressed and energized as the applicator is manipulated from the unprimed configuration to the actuated configuration. The applicator is configured such that axially compressing the actuator and the applicator body manipulates the applicator from the unprimed configuration to the actuated configuration, where manipulating the applicator from the unprimed configuration to the actuated configuration causes the actuator to inwardly deflect the latch and disengage the latch from the slot of the applicator body to release the plunger.

Example Ex2. The applicator of Ex1, where the stored energy device includes a wave spring.

Example Ex3. The applicator of Ex1, where the store energy device includes at least one of a conical coil spring, a coil spring, or a tension spring.

Example Ex4. The applicator of any one of Ex1 to Ex3, where an outer surface of the actuator includes an ergonomic shape.

Example Ex5. The applicator of any one of Ex1 to Ex4, where the actuator further includes an actuator arm extending from an upper portion of the inner surface of the actuator that engages an upper tab of the applicator body to retain the actuator to the applicator body, where the upper tab extends from the lower portion of the applicator body.

Example Ex6. The applicator of any one of Ex1 to Ex5, where the actuator includes a cavity disposed in the inner surface of the actuator along the axis, where the cavity is configured to inwardly deflect the latch and disengage the latch from the slot of the applicator body.

Example Ex7. The applicator of any one of Ex1 to Ex6, where the applicator body further includes a recess disposed in the lower portion of the applicator body that is configured to receive a portion of a lower surface of the actuator.

Example Ex8. The applicator of any one of Ex1 to Ex7, where the upper portion of the applicator body includes an elliptical cross-section in a plane orthogonal to the axis.

Example Ex9. The applicator of any one of Ex1 to Ex7, where the upper portion of the applicator body includes a cylindrical shape.

Example Ex10. The applicator of any one of Ex1 to Ex9, where the lower portion of the applicator body includes an elliptical cross-section in a plane orthogonal to the axis.

Example Ex11. The applicator of any one of Ex1 to Ex10, where the applicator body further includes a rib disposed on an outer surface of the upper portion of the applicator body that extends in a direction substantially parallel to the axis, where the rib is configured to be slidably received by a slot that is disposed in the inner surface of the actuator to prevent the actuator from rotating about the axis when the actuator is manipulated along the axis relative to the applicator body.

Example Ex12. The applicator of any one of Ex1 to Ex11, where the plunger further includes second and third latches extending from the inner surface of the base, where each of the second and third latches is configured to engage the slot disposed in the upper surface of the applicator body to retain the plunger at least partially within the opening of the applicator body when the applicator is in the unprimed configuration, and further where manipulating the actuator from the unprimed configuration to the actuated configuration causes the actuator to inwardly deflect the second and third latches and disengage the second and third latches from the slot of the applicator body to release the plunger.

Example Ex13. The applicator of any one of Ex1 to Ex12, where the plunger further includes an arm extending from the base and configured to engage a protrusion disposed in the opening of the applicator body and retain the plunger to the applicator body.

Example Ex14. The applicator of any one of Ex1 to Ex13, where an outer surface of the base of the plunger is configured to contact the microneedle array and drive the array through a lower surface of the lower portion of the applicator body when the applicator is manipulated from the unprimed configuration to the actuated configuration.

Example Ex15. The applicator of Ex14, where the outer surface of the base of the plunger is configured to be in contact with the microneedle array when the applicator is in the unprimed configuration.

Example Ex16. A system that includes a microneedle array and a microneedle array applicator. The applicator includes an applicator body having an upper portion, a lower portion, and an opening extending through the upper portion and the lower portion along an axis, where the lower portion is configured to be disposed proximate skin of a user; an actuator configured to slidably engage with the applicator body between an unprimed configuration and an actuated configuration of the applicator, a plunger disposed at least partially within the opening of the applicator body. The plunger includes a base and a latch that extends from an inner surface of the base. The latch is configured to engage a slot disposed in the upper portion of the applicator body to retain the plunger at least partially within the opening of the applicator body when the applicator is in the unprimed configuration. The applicator further includes a stored energy device disposed between the base of the plunger and an inner surface of the actuator. The stored energy device is configured to be compressed and energized as the applicator is manipulated from the unprimed configuration to the actuated configuration. The applicator is configured such that axially compressing the actuator and the applicator body manipulates the applicator from the unprimed configuration to the actuated configuration, where manipulating the applicator from the unprimed configuration to the actuated configuration causes the actuator to inwardly deflect the latch and disengage the latch from the slot of the applicator body to release the plunger.

Example Ex17. The system of Ex16, where the microneedle array includes a plurality of microneedles disposed on a substrate, a backing layer, and an adhesive layer disposed on the backing layer between the backing layer and the substrate of the microneedle array.

Example Ex18. The system of any one of Ex 16 to Ex17, where the microneedle array is disposed within the opening of the applicator body of the applicator when the applicator is disposed in the unprimed configuration.

Example Ex19. The system of any one of Ex16 to Ex18, where the microneedle array is retained within the opening of the applicator body by a tab that extends from an inner surface of the lower portion of the applicator body.

Example Ex20. The system of any one of Ex16 to Ex19, where the stored energy device of the applicator includes a wave spring.

Example Ex21. The system of any one of Ex16 to Ex19, where the store energy device of the applicator includes at least one of a conical coil spring, a coil spring, or a tension spring.

Example Ex22. The system of any one of Ex16 to Ex21, where an outer surface of the actuator of the applicator includes an ergonomic shape.

Example Ex23. The system of any one of Ex16 to Ex22, where the actuator of the applicator further includes an actuator arm extending from an upper portion of the inner surface of the actuator that engages an upper tab of the applicator body of the applicator to retain the actuator to the applicator body, where the upper tab extends from the lower portion of the applicator body.

Example Ex24. The system of any one of Ex16 to Ex23, where the actuator includes a cavity disposed in the inner surface of the actuator along the axis, where the cavity is configured to inwardly deflect the latch and disengage the latch from the slot of the applicator body.

Example Ex25. The system of any one of Ex16 to Ex24, where the applicator body of the applicator further includes a recess disposed in the lower portion of the applicator body that is configured to receive a portion of lower surface of the actuator.

Example Ex26. The system of any one of Ex16 to Ex25, where the upper portion of the applicator body includes an elliptical cross-section in a plane orthogonal to the axis.

Example Ex27. The system of any one of Ex16 to Ex25, where the upper portion of the applicator body includes a cylindrical shape.

Example Ex28. The system of any one of Ex16 to Ex27, where the lower portion of the applicator body includes an elliptical cross-section in a plane orthogonal to the axis.

Example Ex29. The system of any one of Ex16 to Ex28, where the applicator body of the applicator further includes a rib disposed on an outer surface of the upper portion of the applicator body that extends in a direction substantially parallel to the axis, where the rib is configured to be slidably received by a slot that is disposed in the inner surface of the actuator to prevent the actuator from rotating about the axis when the actuator is manipulated along the axis relative to the applicator body.

Example Ex30. The applicator of any one of Ex16 to Ex29, where the plunger of the applicator further includes second and third latches extending from the inner surface of the base, where each of the second and third latches is configured to engage the slot disposed in the upper surface of the applicator body to retain the plunger at least partially within the opening of the applicator body when the applicator is in the unprimed configuration, and further where manipulating the actuator from the unprimed configuration to the actuated configuration causes the actuator to inwardly deflect the second and third latches and disengage the second and third latches from the slot of the applicator body to release the plunger.

Example Ex31. The system of any one of Ex16 to Ex30, where the plunger of the applicator further includes an arm extending from the base and configured to engage a protrusion disposed in the opening of the applicator body and retain the plunger to applicator body.

Example Ex32. The system of any one of Ex16 to Ex31, where an outer surface of the base of the plunger of the applicator is configured to contact the microneedle array and drive the microneedle array through a lower surface of the lower portion of the applicator body when the actuator is manipulated from the unprimed configuration to the actuated configuration.

Example Ex33. The system of Ex32, where the outer surface of the plunger is configured to be in contact with the microneedle array when the applicator is in the unprimed configuration.

Example Ex34. A method that includes disposing a microneedle array within an opening in an applicator body of an applicator, where the opening extends through an upper portion and a lower portion of the applicator body along an axis, and where the applicator further includes an actuator slidably engaged with the applicator body and a plunger disposed at least partially within the opening of the applicator body. The method further includes disposing the applicator proximate skin of a user, and manipulating the applicator from an unprimed configuration to an actuated configuration. Manipulating the applicator from the unprimed configuration to the actuated configuration includes axially compressing the actuator of the applicator and the applicator body along the axis. A stored energy device disposed between a base of the plunger and an inner surface of the actuator is compressed and energized when the applicator is manipulated from the unprimed configuration to the actuated configuration. Further, the actuator inwardly defects a latch that extends from an inner surface of a base of the plunger and disengages the latch from a slot disposed in the upper portion of the applicator body to release the plunger.

Example Ex35. The method of Ex34, where manipulating the applicator from the unprimed configuration to the actuated configuration causes the plunger to drive the microneedle array towards the skin of the user such that the microneedle array is delivered to the skin.

Example Ex36. The method of any one of Ex34 to Ex35, further including removing the applicator from proximate the skin of the user after the applicator has been manipulated to the actuated configuration.

Example Ex37. The method of any one of Ex34 to Ex36, where the microneedle array includes a plurality of microneedles disposed on a substrate, a backing layer, and an adhesive layer disposed on the backing layer between the backing layer and the substrate of the microneedle array.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1-20. (canceled)

21. A microneedle array applicator comprising:
an applicator body comprising an upper portion, a lower portion, and an opening extending through the upper portion and the lower portion along an axis, wherein the lower portion is configured to be disposed proximate skin of a user;
an actuator configured to slidably engage with the applicator body between an unprimed configuration and an actuated configuration of the applicator;
a plunger disposed at least partially within the opening of the applicator body, wherein the plunger comprises a base and a latch that extends from an inner surface of the base, wherein the latch is configured to engage a slot disposed in the upper portion of the applicator body to retain the plunger at least partially within the opening of the applicator body when the applicator is in the unprimed configuration; and
a stored energy device disposed between the base of the plunger and an inner surface of the actuator, wherein the stored energy device is configured to be compressed and energized as the applicator is manipulated from the unprimed configuration to the actuated configuration;
wherein the applicator is configured such that axially compressing the actuator and the applicator body manipulates the applicator from the unprimed configuration to the actuated configuration, wherein manipulating the applicator from the unprimed configuration to the actuated configuration causes the actuator to inwardly deflect the latch and disengage the latch from the slot of the applicator body to release the plunger.

22. The applicator of claim 21, wherein the stored energy device comprises at least one of a wave spring, a conical coil spring, a coil spring, or a tension spring.

23. The applicator of claim 21, wherein the actuator further comprises an actuator arm extending from an upper portion of the inner surface of the actuator that engages an upper tab of the applicator body to retain the actuator to the applicator body, wherein the upper tab extends from the lower portion of the applicator body.

24. The applicator of claim 21, wherein the actuator comprises a cavity disposed in the inner surface of the actuator along the axis, wherein the cavity is configured to inwardly deflect the latch and disengage the latch from the slot of the applicator body.

25. The applicator of claim 21, wherein the applicator body further comprises a recess disposed in the lower portion of the applicator body that is configured to receive a portion of a lower surface of the actuator.

26. The applicator of claim 21, wherein the applicator body further comprises a rib disposed on an outer surface of the upper portion of the applicator body that extends in a direction substantially parallel to the axis, wherein the rib is configured to be slidably received by a slot that is disposed in the inner surface of the actuator to prevent the actuator from rotating about the axis when the actuator is manipulated along the axis relative to the applicator body.

27. The applicator of claim 21, wherein the plunger further comprises second and third latches extending from the inner surface of the base, wherein each of the second and third latches is configured to engage the slot disposed in the upper surface of the applicator body to retain the plunger at least partially within the opening of the applicator body when the applicator is in the unprimed configuration, and further wherein manipulating the actuator from the unprimed configuration to the actuated configuration causes the actuator to inwardly deflect the second and third latches and disengage the second and third latches from the slot of the applicator body to release the plunger.

28. The applicator of claim 21, wherein the plunger further comprises an arm extending from the base and configured to engage a protrusion disposed in the opening of the applicator body and retain the plunger to the applicator body.

29. The applicator of claim 21, wherein an outer surface of the base of the plunger is configured to contact the microneedle array and drive the array through a lower surface of the lower portion of the applicator body when the applicator is manipulated from the unprimed configuration to the actuated configuration.

30. The applicator of claim 29, wherein the outer surface of the base of the plunger is configured to be in contact with the microneedle array when the applicator is in the unprimed configuration.

31. A system comprising a microneedle array and a microneedle array applicator, wherein the applicator comprises:
an applicator body comprising an upper portion, a lower portion, and an opening extending through the upper portion and the lower portion along an axis, wherein the lower portion is configured to be disposed proximate skin of a user;
an actuator configured to slidably engage with the applicator body between an unprimed configuration and an actuated configuration of the applicator;
a plunger disposed at least partially within the opening of the applicator body, wherein the plunger comprises a base and a latch that extends from an inner surface of the base, wherein the latch is configured to engage a slot disposed in the upper portion of the applicator body to retain the plunger at least partially within the opening of the applicator body when the applicator is in the unprimed configuration; and
a stored energy device disposed between the base of the plunger and an inner surface of the actuator, wherein the stored energy device is configured to be compressed and energized as the applicator is manipulated from the unprimed configuration to the actuated configuration;
wherein the applicator is configured such that axially compressing the actuator and the applicator body manipulates the applicator from the unprimed configuration to the actuated configuration, wherein manipulating the applicator from the unprimed configuration to the actuated configuration causes the actuator to inwardly deflect the latch and disengage the latch from the slot of the applicator body to release the plunger.

32. The system of claim 31, wherein the microneedle array comprises:
a plurality of microneedles disposed on a substrate;
a backing layer; and
an adhesive layer disposed on the backing layer between the backing layer and the substrate of the microneedle array.

33. The system of any claim 31, wherein the microneedle array is disposed within the opening of the applicator body of the applicator when the applicator is disposed in the unprimed configuration.

34. The system of claim 31, wherein the microneedle array is retained within the opening of the applicator body by a tab that extends from an inner surface of the lower portion of the applicator body.

35. The system of claim 31, wherein the actuator of the applicator further comprises an actuator arm extending from an upper portion of the inner surface of the actuator that engages an upper tab of the applicator body of the applicator to retain the actuator to the applicator body, wherein the upper tab extends from the lower portion of the applicator body.

36. The system of claim 31, wherein the actuator comprises a cavity disposed in the inner surface of the actuator along the axis, wherein the cavity is configured to inwardly deflect the latch and disengage the latch from the slot of the applicator body.

37. The system of claim 31, wherein the applicator body of the applicator further comprises a recess disposed in the lower portion of the applicator body that is configured to receive a portion of lower surface of the actuator.

38. The applicator of claim 31, wherein the plunger of the applicator further comprises second and third latches extending from the inner surface of the base, wherein each of the second and third latches is configured to engage the slot disposed in the upper surface of the applicator body to retain the plunger at least partially within the opening of the applicator body when the applicator is in the unprimed configuration, and further wherein manipulating the actuator from the unprimed configuration to the actuated configuration causes the actuator to inwardly deflect the second and third latches and disengage the second and third latches from the slot of the applicator body to release the plunger.

39. A method comprising:
disposing a microneedle array within an opening in an applicator body of an applicator, wherein the opening extends through an upper portion and a lower portion of the applicator body along an axis, wherein the applicator further comprises an actuator slidably engaged with the applicator body and a plunger disposed at least partially within the opening of the applicator body;
disposing the applicator proximate skin of a user; and
manipulating the applicator from an unprimed configuration to an actuated configuration, wherein manipulating the applicator from the unprimed configuration to the actuated configuration comprises axially compressing the actuator of the applicator and the applicator body along the axis, wherein a stored energy device disposed between a base of the plunger and an inner surface of the actuator is compressed and energized when the applicator is manipulated from the unprimed configuration to the actuated configuration, wherein the actuator inwardly defects a latch that extends from an inner surface of a base of the plunger and disengages the latch from a slot disposed in the upper portion of the applicator body to release the plunger.

40. The method of claim 39, wherein manipulating the applicator from the unprimed configuration to the actuated configuration causes the plunger to drive the microneedle array towards the skin of the user such that the microneedle array is delivered to the skin.
